(19) United States
(12) Patent Application Publication
(10) Pub. No.: US 2007/0082068 A1
(43) Pub. Date: Apr. 12, 2007

(54) Il-8 production promoters and use thereof

(76) Inventors: Hideo Satsu, Tokyo (JP); Tatsumasa Mae, Hyogo (JP); Misuzu Tsukagawa, Hyogo (JP); Mikio Kitahara, Hyogo (JP); Shinichi Yokota, Hyogo (JP); Makoto Shimizu, Tokyo (JP)

Correspondence Address: BRINKS HOFER GILSON & LIONE, P.O. BOX 10395, CHICAGO, IL, 60610, US

(21) Appl. No.: 10/579,323

(22) PCT Filed: Nov. 17, 2004
(86) PCT No.: PCT/JP04/17443

§ 371 Date: May 16, 2006

(30) Foreign Application Priority Data
Nov. 25, 2003 (JP) 2003-394695

(51) Int. Cl. A61K 36/185; A61K 36/534; A61K 31/045; A61K 31/015; A61K 36/48

(52) U.S. Cl. 424/725; 424/747; 424/757; 514/763; 514/729

(57) ABSTRACT

An object of the present invention is to provide IL-8 production promoters derived from safe food materials and to provide immunostimulants or preventive or treating agents of infectious disease comprising the IL-8 production promoter. The present inventors have found that a composition comprising, as an active ingredient, at least one plant extract from peppermint, dokudami (houttuynia herb), or licorice promotes IL-8 production, and that a composition comprising, as an active ingredient, at least one compound selected from the group consisting of α-humulene, α-pinene, β-pinene, and L-menthol promotes IL-8 production. These IL-8 production promoters can be used in foods and drinks supplemented with immunostimulants or preventive or treating agents of infectious disease.

### TECHNICAL FIELD

The present invention relates to interleukin-8 production promoters and to immunostimulants or preventive or treating agents of infectious disease comprising this interleukin-8 production promoter.

### BACKGROUND ART

Intestinal tract directly comes into contact with various types of foods and so on taken orally, and therefore has both of two vital functions, the function of eliminating harmful substances and the function of absorbing useful components. While the physiological functions of foods are exhibited mainly by substance absorption in intestinal tract and by signal transduction or cytokine regulation brought about by the absorbed components via cells, intestinal immunity among those functions of the intestinal tract plays a key role as a front-line immune system in vivo. The intestinal tract has immunocompetent tissue (GALT: gut associated lymphoreticular tissue) that induces immune reactions against orally ingested antigens as well as intestinal epithelial cells that absorb useful components such as nutrients from foods. Food components act directly or indirectly on these cells and thereby perform immunoregulation, that is, immune promotion or suppression.

Intestinal epithelial cells are known to produce cytokines such as TGF-β, IL-1β, IL-10, and TNF-α, and chemokines such as IL-8, and lead to a increase in the chemokines by infection with a variety of pathogenic bacteria. The intestinal epithelial cells interact with immunocompetent cells through cytokines or chemokines. Among others, IL-8 is deeply implicated in host defense, immune systems, and inflammatory response. Interleukin-8 (hereinafter, abbreviated to IL-8), a neutrophil chemotactic/activating factor, was purified and cloned in 1987 from human peripheral blood mononuclear cells stimulated with LPS (Proc. Natl. Acad. Sci. USA, 84, 9233-9237, 1987).

IL-8 is produced by monocytes, macrophages, fibroblasts, vascular endothelial cells, mast cells, epidermal cells, and the like, and targets cells such as neutrophils, CD8+ T cells, natural killer cells, and monocytes. The known functions of IL-8 include: neutrophil chemotaxis; chemotactic activity for basophils and T-cells; the mobilization of neutrophils from the bone marrow to peripheral blood; neutrophil activation such as release of intracellular lysosomal enzyme, induction of leukotrien B₄ (LTB₄) production, and induction of active oxygen production, in neutrophils; enhancement of neutrophil adherence to vascular endothelial cells; neutrophil chemotactic activity for human umbilical vein endothelial cells (HUVEC) ; and involvement in vascularization (Cell Technology Suppl., Chemokine Handbook, Vol. 1, p. 32-34, Shujunsya, 2000).

Alternatively, it is known that neutrophils are leukocytes having a central role in host defense by virtue of their phagocytic abilities to engulf and kill invading bacteria (e.g., Escherichia coli, staphylococci, streptococci, and pneumococci), viruses, and fungi, and IL-8 promotes neutrophil activation and enhances abilities of neutrophils.

The action of aggressive immune enhancement through ingestion of foods imparts resistance to living bodies, leading to prevention or treatment of infectious disease. Moreover, cytokines and so on that are involved in immune enhancement provide for therapeutic effects on immune disease such as allergy and for antitumor effects. Although absorption and permeation of food components in intestinal tract have been reported previously, reports that have directly demonstrated the influence of food components on intestinal epithelial cells are few, which include the inhibition of IL-8 production by curcumin (J. Immunol., 163, 3474, 1999), and the promotion of IL-8 production by means of the oral administration of microbial cells such as lactic acid bacteria (Japanese Patent Laid-Open No. 2003-63991).

Alternatively, bacterial invasion to intestinal epithelial cells has been reported to increase IL-8production (J. Clin. Invest., 95, 55-65, 1995). Since IL-8 is closely related to host defense, immunity, and inflammatory response, the promotion of IL-8 production in intestinal epithelial cells can be useful particularly in aggressive immunostimulation and in the prevention or treatment of infectious disease.

It is an unknown fact that peppermint or extracts thereof, dokudami (houttuynia herb) or extracts thereof, and licorice or extracts thereof promote IL-8 production or are useful in immunostimulation or in the prevention or treatment of infectious disease, to which IL-8 contributes.

Moreover, it is also an unknown fact that (α-humulene, pinene, and L-menthol promote IL-8 production or are useful in immunostimulation or in the prevention or treatment of infectious disease, to which IL-8 contributes.

### DISCLOSURE OF THE INVENTION

IL-8 production promoters derived from safe food materials are useful in stimulation of immune response and in prevention or treatment of infectious disease, for example as foods and drinks such as foods with health claims (foods for specified health uses, foods with nutrient function claims), health foods, and dietary supplements, or as pharmaceuticals or quasi drugs. However, it is difficult to continuously ingest chemokines such as IL-8 for the purpose of activating neutrophils to enhance their phagocytic abilities. Accordingly, an object of the present invention is to provide IL-8 production promoters derived from safe food materials and to provide immunostimulants or preventive or treating agents of infectious disease comprising this IL-8 production promoter.

The present inventors have conducted extensive studies for attaining the object and have consequently completed the present invention by finding that a composition comprising, as an active ingredient, at least one plant extract from peppermint, dokudami (houttuynia herb), or licorice promotes IL-8production, and that a composition comprising as an active ingredient at least one compound selected from the group consisting of α-humulene, α-pinene, β-pinene, and L-menthol promotes IL-8 production.

Namely, the present invention relates to an IL-8 production promoter comprising a peppermint extract and/or a dokudami (houttuynia herb) extract as an active ingredient.

The present invention also relates to an IL-8 production promoter comprising a licorice extract as an active ingredient.

Moreover, the present invention relates to an interleukin-8 production promoter comprising as an active ingredient at least one compound selected from the group consisting of α-humulene represented by formula (1): 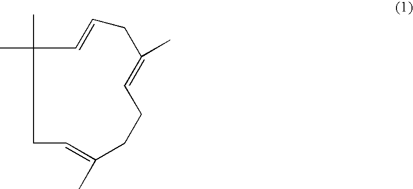
α-pinene represented by formula (2): 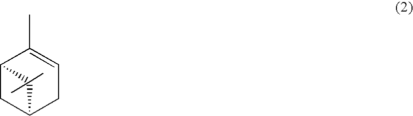
β-pinene represented by formula (3): 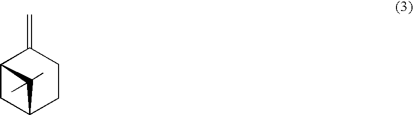
and L-menthol represented by formula (4): 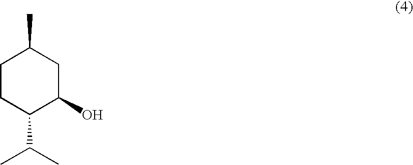
or a pharmaceutically acceptable salt thereof.

### BEST MODE FOR CARRYING OUT THE INVENTION

Hereinafter, the embodiments of the present invention will be described in detail.

In one aspect, IL-8 production promoters of the present invention comprise, as an active ingredient, at least one plant extract from peppermint, dokudami (houttuynia herb), or licorice. The IL-8 described herein refers to a leukocyte chemotactic factor with a molecular weight of approximately 8,000 that is produced by a variety of cells such as fibroblasts and endothelial cells.

Peppermint used in the present invention refers to an herb that belongs to genus Mentha of family Lamiacea (Mentha piperita L, and Mentha arvensis). Dokudami (houttuynia herb), also called “jyuyaku”, used in the present invention refers to an above-ground part (e.g., leaves, stems, and flowers) of Houttuynia cordata Thunb. belonging to genus Houttuynia of family Saururaceae. Licorice used in the present invention refers to a rhizome of Glycyrrhiza uralensis Fish. belonging to genus Glycyrrhiza of family Leguminosae. All of these plants are foods or food materials and have sufficient history of use as foods, without problems associated with side effects and safety.

Each extract from peppermint, dokudami (houttuynia herb), and licorice used in the present invention can be obtained from the plants by solvent extraction or the like. A method for obtaining the extract is not limited to the solvent extraction, and other extraction procedures may be used, which include steam distillation and a supercritical extraction technique using carbon dioxide. Furthermore, the extract, unless containing impurities inadequate for foods and drinks or pharmaceutical drugs, can be used in the present invention, either directly in extracted solution form or as a crude or semi-purified extract.

The production method of the extract is preferably performed under room temperature and normal pressures using an extraction solvent. After extraction, the resulting product may be made into a liquid, paste, gel, or powder by means of concentration and drying or with use of fats and oils or the like. An extraction temperature is not particularly limited and is generally performed under a condition of −20 to 100° C., preferably 1 to 80° C., more preferably 20 to 60° C. Likewise, an extraction time is not particularly limited, and the extraction is generally performed with stirring or by leaving the starting material to stand for 0.1 to 1 month, preferably 0.5 hours to 7 days. Alternatively, the extraction may be performed under supercritical conditions using carbon dioxide or the like. The extract can optionally be purified by arbitrary procedures using active carbon treatment, ion-exchange resins, and so on.

When solvent extraction is performed, for example, an original, crushed or powder form of each of the plants can be dipped in a 1 to 20-fold volume of a solvent described below and stirred or left to stand, followed by filtration or centrifugation to obtain an extracted solution. Subsequently, the solvent can be removed from the obtained extracted solution by concentration to obtain the extract.

The solvent used in extraction is not particularly limited, and for example, a hydrophilic or hydrophobic solvent can be used. In this context, the hydrophilic solvent refers to a solvent with high polarity and includes water, lower alcohol such as ethanol and methanol, polyhydric alcohol such as propylene glycol, and ketones such as acetone. In this context, the hydrophobic solvent refers to a nonpolar solvent with low polarity and includes esters such as ethyl acetate, fats and oils such as rapeseed oil and olive oil, and hydrocarbons such as hexane. It is preferred that the solvent should be a safe substance available in production or processing of foods, food additives, pharmaceuticals, and so on. Examples of such a solvent include water, ethanol, acetone, glycerin, ethyl acetate, propylene glycol, hexane, and edible fats and oils. It is more preferable to use one or two or more solvents selected from the group consisting of water, ethanol, acetone, glycerin, ethyl acetate, propylene glycol, hexane, and edible fats and oils. The solvent such as ethanol, acetone, ethyl acetate, and hexane is more preferable from the viewpoint of extraction efficiency and ease of removing the solvent after extraction. It is even more preferable to use one or two or more solvents selected from the group consisting of ethanol, acetone, ethyl acetate, and hexane. Ethanol is most preferable from the viewpoint of safety of the residual solvent. The hydrophilic solvent may be used in the form of a hydrous solvent.

Among the extracts thus obtained, a peppermint extract obtained from peppermint herbs by steam distillation or solvent extraction is a kind of spice extract and is an existing additive applied to bitter agents and so on. Moreover, a dokudami (houttuynia herb) extract obtained from dokudami (houttuynia herb) leaves by ethanol extraction and purification is an existing additive applied to antioxidants. Furthermore, a licorice extract obtained from the licorice roots by ethanol extraction is an existing additive applied to antioxidants. In the present invention, these peppermint, dokudami (houttuynia herb), and licorice extracts accepted as food additives can also be used.

In one aspect, the present invention provides IL-8 production promoters comprising as an active ingredient at least one compound selected from the group consisting of peppermint component compounds, that is, α-humulene represented by formula (1) below, α-pinene represented by formula (2) below, β-pinene represented by formula (3) below, and L-menthol represented by formula (4) below, or a pharmaceutically acceptable salt thereof. The influence of the component compounds contained in peppermint on IL-8 secretion by Caco-2 cells was examined. As a result, α-humulene represented by formula (1) below, α-pinene represented by formula (2) below, β-pinene represented by formula (3) below, and L-menthol represented by formula (4) below were shown to have the effect of promoting IL-8 production.

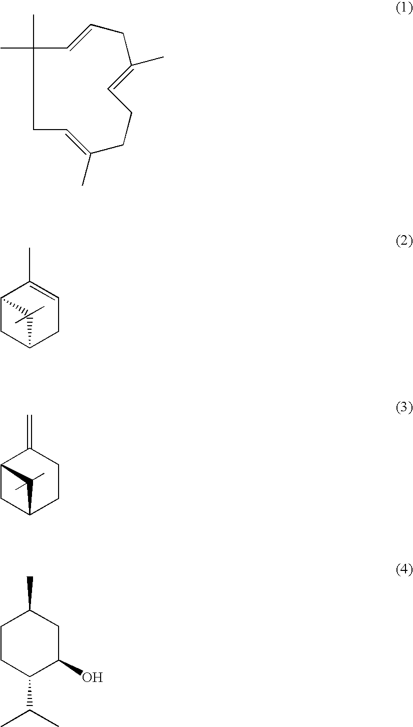

A method for obtaining the α-humulene, α-pinene, β-pinene, and L-menthol represented by formulas (1) to (4) is not particularly limited. The obtained compounds can be used as single compounds or as mixtures of two or more of them in the IL-8 production promoters of the present invention. When these compounds are obtained from peppermint, the same extraction methods as above can be employed. After extraction, the resulting products can be subjected to additional procedures such as separation and purification, for example, filtration, distribution using solvents, concentration, distillation, steam distillation, chromatography, to obtain the compounds. It is preferred that extraction solvents, purification resin, tools, apparatuses, and so on, used in the procedures should be available for use in the production of foods or food additives. Types of plants from which α-humulene, α-pinene, β-pinene, and L-menthol are extracted are not particularly limited, and include cloves (Syzygium aromaticum) of the family Myrtaceae and hops (Humulus lupulus) and so on.

Salts of these compounds are also preferably available in the present invention. When the compounds have a basic moiety, the salts of the compounds can be formed by mixing solutions of the compounds with a solution of a pharmaceutically acceptable acid, for example, hydrochloric acid, sulfuric acid, methanesulfonic acid, fumaric acid, maleic acid, succinic acid, acetic acid, benzoic acid, oxalic acid, citric acid, tartaric acid, carbonic acid, orphosphoric acid. When the compounds have an acidic moiety, their pharmaceutically acceptable salts include salts formed with alkali metal salts such as sodium or potassium salts; alkaline earth metal salts such as calcium or magnesium salts; and salts formed with suitable organic ligands such as quaternary ammonium salts.

The IL-8 production promoters are capable of stimulating immunity in mammal or preventing or treating the infectious disease of mammal. The “immunostimulation” or “stimulating immunity” described herein refers to the activation of immunocytes such as leukocytes that are responsible for the immunological function of eliminating “not-self” such as invading foreign substances (such as bacteria and viruses) or cancer cells. The infectious disease described herein refers to food poisoning and disease caused by, for example, respiratory tract infection via droplets, vehicle-borne infection via water and foods, vector-borne infection via insects and animals, oral infection via foods and drinks, opportunistic infection with harmless indigenous bacteria or attenuated bacteria during the suppression of immunity. Examples of the infectious disease include respiratory infection disease, urinary tract infectious disease, and septicemia, and further include viral disease, bacterial disease, and AIDS.

In one aspect, the present invention provides compositions comprising the IL-8 production promoter as an active ingredient and supplemented with carriers, auxiliaries, food or drink materials, other pharmaceutically acceptable pharmaceutical materials, and so on known in the art. The extract of the present invention from at least one plant selected from the group consisting of peppermint, dokudami (houttuynia herb), and licorice promotes the IL-8 production of mammal including human beings. Moreover, the compositions for promoting IL-8 production of the present invention promote IL-8 production and as such, can be used as compositions for promoting immunostimulants or for prevention or treatment of infectious disease, to which IL-8 contributes.

The compositions provided by the present invention comprise, as an active ingredient, preferably 0.1 to 100% by weight, more preferably 1 to 100% by weight, of at least one kind selected from a group consisting of a peppermint extract, a dokudami (houttuynia herb) extract, a licorice extract, a compound represented by formula (1), a compound represented by formula (2), a compound represented by formula (3), a compound represented by formula (4), and pharmaceutically acceptable salts thereof. The compositions comprise even more preferably 10 to 100% by weight, still more preferably 20 to 100% by weight, most preferably 30 to 100% by weight, of the active ingredient.

A peppermint extract, a dokudami (houttuynia herb) extract, a licorice extract, α-humulene, α-pinene, β-pinene, and L-menthol used in the present invention can be evaluated for their effects of promoting IL-8 production by a method that adds or administers the extracts or compounds to an experimental system that produces IL-8. For example, in an in-vitro experimental system, human colon cancer-derived cell line HT-29 or Caco-2 cells secrete IL-8 into a medium and therefore, can be cultured in the medium supplemented with the extracts to evaluate the effects of promoting IL-8 production by quantifying the amount of IL-8 secreted in the medium. Alternatively, the effects of promoting IL-8 production can be evaluated in a similar experimental system using these cells stimulated with TNF-α in a manner that enhances the amount of IL-8 produced (Chowers, Y., et al., Gastroenterology, 120, 449-459, 2001; and Lahav, M., et al., Clin. Exp. Immunol., 127, 226-233, 2002). Specifically, tests are conducted as described in Examples below to assess significant components at a significant level: P<0.01 as having the effect of promoting IL-8 production.

The IL-8 production promoters, immunostimulants, and treatment or improvement agents of infectious disease of the present invention, and the compositions comprising any one of them as an active ingredient (hereinafter, referred to as the preventive or treating agents and compositions of the present invention) can be utilized for food and drink use and medicinal purposes. The forms are not limited, and the preventive or treating agents and compositions of the present invention can be used, for example as foods and drinks such as foods with health claims (foods for specified health uses, foods with nutrient function claims), health foods, and foods and drinks such as dietary supplements, or as easily available pharmaceutical drugs such as over-the-counter drugs, or as quasi drugs.

The preventive or treating agents and compositions of the present invention may contain components other than each extract from peppermint, dokudami (houttuynia herb), and licorice. Since each extract from peppermint, dokudami (houttuynia herb), and licorice conventionally applied to foods, or a compound contained in the extract serves as an active ingredient, the extracts or compounds of the present invention are highly safe for living bodies. Therefore, the IL-8 production promoters of the present invention can be contained in a wide range of products such as pharmaceutical drugs, functional foods, foods and drinks, and quasi drugs.

The content of the active ingredient in the preventive or treating agents and compositions of the present invention is not particularly specified and can be selected appropriately from within a range that exerts desired effects. When the preventive or treating agents and compositions of the present invention are administered to mammal, for example, human body, the dosage thereof is preferably 0.001 to 1000 mg/kg of body weight/day, more preferably 0.01 to 100 mg/kg of body weight/day, in terms of the amount of the active ingredient, in one dose or several doses.

When the preventive or treating agents and compositions of the present invention are used as foods and drinks, they can be ingested either directly or after they are made into preparations in easily taken forms such as capsules, tablets, or granules with use of additives such as carriers and auxiliaries known in the art. The content of the active ingredient (extract) in these preparations is preferably 0.01 to 100% by weight, more preferably 0.1 to 90% by weight. Types of intended foods and drinks are not particularly limited, unless they inhibit the effects of promoting IL-8 production exhibited by the extracts.

For example, the preventive or treating agents and compositions of the present invention are mixed with food and drink materials and can thereby be used in all kinds of foods and drinks including: confectionery such as chewing gums, chocolates, candies, jellies, biscuits, and crackers; frozen desserts such as ice creams and sherbets; drinks such as tea, soft drinks, energy drinks, and beauty drinks; noodles such as Japanese wheat noodles, Chinese noodles, spaghetti, and instant noodles; fish cake or processed marine products such as kamaboko (steamed fish paste), chikuwa (tubular fish cake), hanpen (fish minced and steamed); seasonings such as dressing, mayonnaise, and sauce; fats and oils such as margarine, butter, and cooking oil , and bread, hams, soup, retort pouch foods, and frozen foods.

When the preventive or treating agents and compositions of the present invention are ingested for food and drink use, the daily intake thereof in adult is preferably 0.001 to 1000 mg/kg of body weight, more preferably 0.01 to 100 mg/kg of body weight, in terms of the amount of the active ingredient.

When the preventive or treating agents and compositions of the present invention are processed into preparations for use as pharmaceutical drugs, the dosage forms are not particularly limited and can be tablets, capsules, injections, drops, powders, suppositories, granules, ointments, suspensions, emulsions, syrups, creams, or the like, selected depending on the purpose of administration, administration routes, and so on. These compositions can contain additives generally used in preparations such as binders, excipients, lubricants, disintegrants, stabilizers, emulsifiers, and buffers. Preferable examples of the binders include starch, trehalose, dextrin, and powders of gum arabic. Preferable examples of the excipients include white sugar, milk sugar, grape sugar, cornstarch, mannitol, crystalline cellulose, calcium phosphate, and calcium sulfate. Preferable examples of the lubricants include stearic acid, talc, wax, and polyethylene glycol. Preferable examples of the disintegrants include starch, carboxymethylcellulose, and cornstarch. Preferable examples of the stabilizers include fats and oils and propylene glycol. Preferable examples of the emulsifiers include anionic surfactants, nonionic surfactants, and polyvinyl alcohol. Preferable examples of the buffers include phosphate, carbonate, and citrate buffer solutions. The daily dosage of these preparations in adult is preferably 0.001 to 1000 mg/kg of body weight, more preferably 0.01 to 100 mg/kg of body weight, in terms of the amount of the extract, in one dose or several doses.

When the preventive or treating agents and compositions of the present invention are processed into preparations for use as quasi drugs, they are optionally supplemented with other additives and the like and can thereby be employed locally as, for example, ointments, liniments, aerosols, cream, soap, face washes, body washes, skin toners, lotions, and bath agents.

In one aspect, the present invention provides a method for promoting interleukin production in mammal, for stimulating immunity in mammal, or for preventing or treating an infectious disease of mammal, comprising administering an effective amount of at least one kind selected from the group consisting of a peppermint extract, a dokudami (houttuynia herb) extract, a licorice extract, a compound represented by formula (1), a compound represented by formula (2), a compound represented by formula (3), a compound represented by formula (4), and a pharmaceutically acceptable salt thereof to mammal that need them. The administration includes enteral administration (e.g., oral administration) and nonenteral administration (e.g., percutaneous administration). Oral administration is preferred. The mammal includes human beings.

### BRIEF DESCRIPTION OF THE DRAWINGS

FIG. 1 shows IL-8 concentrations in the culture supernatants of Caco-2 cells cultured with extracts;

FIG. 2 shows IL-8 concentrations in the culture supernatants of Caco-2 cells cultured with components;

FIG. 3 shows the cytotoxicity of the extracts; and

FIG. 4 shows the cytotoxicity of α-humulene.

### BEST MODE FOR CARRYING OUT THE INVENTION

Hereinafter, the present invention will be described more fully with reference to Examples. However, the scope of the present invention is not intended to be limited to these Examples.

### EXAMPLE 1

### Preparation of Peppermint Extract

In a glass container, 20 g of powder of a peppermint (Mentha piperita L.) herb (Kaneka Sun Spice Co., Ltd.) was dipped in 100 ml of ethanol and left with occasional stirring at room temperature for 1 week under shade conditions. The residual powder was removed by filtration using a filter paper (ADVANTEC No. 2) , and the solvent was removed from the resulting extracted solution by concentration under reduced pressure to obtain 1.30 g of peppermint extract.

### EXAMPLE 2

### Preparation of Dokudami (Houttuynia Herb) Extract

In a glass container, 20 g of powder of the above-ground part of dokudami (houttuynia herb) (Houttuynia cordata Thunb.) (Kaneka Sun Spice Co., Ltd.) was dipped in 100 ml of ethanol and left with occasional stirring at room temperature for 1 week under shade conditions. The residual powder was removed by filtration using a filter paper (ADVANTEC No. 2), and the solvent was removed from the resulting extracted solution by concentration under reduced pressure to obtain 1.11 g of a dokudami (houttuynia herb) extract.

### EXAMPLE 3

### Preparation of Licorice Extract

In a glass container, 20 g of powder of a licorice (Glycyrrhiza uralensis Fish.) rhizome (Kaneka Sun Spice Co., Ltd.) was dipped in 100 ml of ethanol and left with occasional stirring at room temperature for 1 week under shade conditions. The residual powder was removed by filtration using a filter paper (ADVANTEC No. 2), and the solvent was removed from the resulting extracted solution by concentration under reduced pressure to obtain 1.88 g of licorice extract.

### EXAMPLE 4

### Effect of Promoting IL-8 Production (1)

Caco-2 cells (human colon cancer-derived cell line; American Type Culture Collection, Rockville, Md., U.S.A.) were used as small-intestinal epithelial cell models to evaluate the influence of peppermint, dokudami (houttuynia herb), and licorice extracts on IL-8 production by the cells.

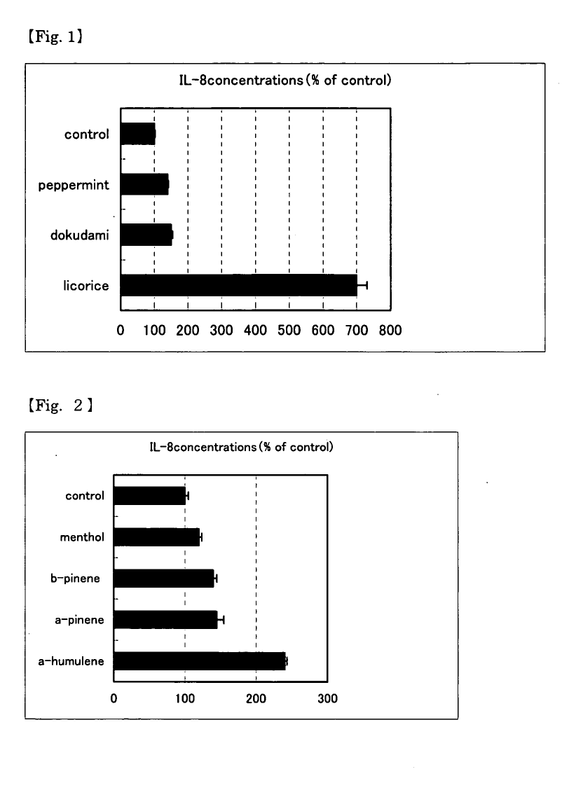

The Caco-2 cells were subcultured in 24-well plates. The resulting cells were cultured at 37° C. for 14 days under 5% CO₂ conditions and thereby differentiated into small-intestinal epithelioid cells. Culture media used were DMEM media (SIGMA Corp.) containing 10% fetal calf serum, 1% non essential amino acid solution, 2% glutamine, 100 U/ml penicillin, and 100 μg/ml streptomycin. A peppermint extract obtained in Example 1, a dokudami (houttuynia herb) extract obtained in Example 2, and a licorice extract obtained in Example 3 were added at each final concentration of 100 μg/ml to the media, and the cells were cultured therein. After 6-hour culture, the media were replaced with additive-free media, followed by additional culture for 12 hours. The resulting culture supernatants were subjected to sandwich ELISA assay (Eckmann, L., et al., Gastroenterology, 105, 1689-1697, 1993) to quantify the amount of IL-8 in the culture supernatants. The relative IL-8 concentrations to a control are shown in FIG. 1.

As seen from FIG. 1, each of the peppermint, dokudami (houttuynia herb), and licorice extracts remarkably promoted IL-8 production by the Caco-2 cells, with a significant difference: P<0.01.

### EXAMPLE 5

### Effect of Promoting IL-8 Production (2)

Caco-2 cells (human colon cancer-derived cell line; American Type Culture Collection, Rockville, Md., U.S.A.) were used as small-intestinal epithelial cell models to evaluate the influence of α-humulene, α-pinene, β-pinene and L-menthol on IL-8 production by the cells.

The Caco-2 cells were subcultured in 24-well plates. The resulting cells were cultured at 37° C. for 14 days under 5% Co₂ conditions and thereby differentiated into small-intestinal epithelioid cells. Culture media used were DMEM media (SIGMA Corp.) containing 10% fetal calf serum, 1% non essential amino acid solution, 2% glutamine, 100 U/ml penicillin, and 100 μg/ml streptomycin. α-humulene (SIGMA Corp.), α-pinene (SIGMA Corp.), β-pinene (SIGMA Corp.), and L-menthol (SIGMA Corp.) were added at each final concentration of 100 μg/ml to the media, and the cells were cultured therein. After 6-hour culture, the media was replaced with additive-free media, followed by additional culture for 12 hours. The resulting culture supernatants were subjected to sandwich ELISA assay to quantify the amount of IL-8 in the culture supernatants. The relative IL-8 concentrations to a control are shown in FIG. 2.

As seen from FIG. 2, the α-humulene, α-pinene, β-pinene, and L-menthol remarkably promoted IL-8 production by the Caco-2 cells, with a significant difference: P<0.01.

### EXAMPLE 6

### Evaluation of Cytotoxicity by LDH (Lactate Dehydrogenase) Assay (1)

The cytotoxicity of peppermint, dokudami (houttuynia herb), licorice extracts was evaluated with Caco-2 cells. The Caco-2 cells differentiated into small-intestinal epithelioid cells in the same way as in Example 4 were cultured for 24 hours in media containing the peppermint, dokudami (houttuynia herb), and licorice extracts at each final concentration of 100 μg/ml. Subsequently, the media were removed, and the resulting cells were washed twice with PBS (phosphate buffered saline) (−) (Nissui Co., Ltd.). The cells were further supplemented with 700 μl of PBS (−) and left undisturbed at 37° C. for 2 hours. Then, the PBS (−) was collected, and the cells were dissolved in 500 μl of 0.1% Triton X-100 (Nacalai Tesque, Inc.). LDH-CytotoxicTestWako (Wako Pure Chemical Industries, Ltd.) was used to measure LDH concentrations in the collected PBS (−) and cell lysate. The principles of the assay are described in detail in Decker, T. and Lohmann-Matthes, M. L., J. Immunol. Methods, 115, 61-69 (1988). In summary, this assay is a method for quantifying cytotoxicity based on the activity of LDH released from dead cells by colormetrically determining formazan converted from a tetrazolium salt serving as a coloring reagent. The rate of free LDH (%) was calculated according to the equation below by using the LDH concentration in the collected PBS (−) as the amount of free LDH and the LDH concentration in the cell lysate as the amount of intracellular LDH.
Rate of free LDH(%)=(Amount of free LDH)/(Amount of free LDH+Amount of intracellular LDH)×100.

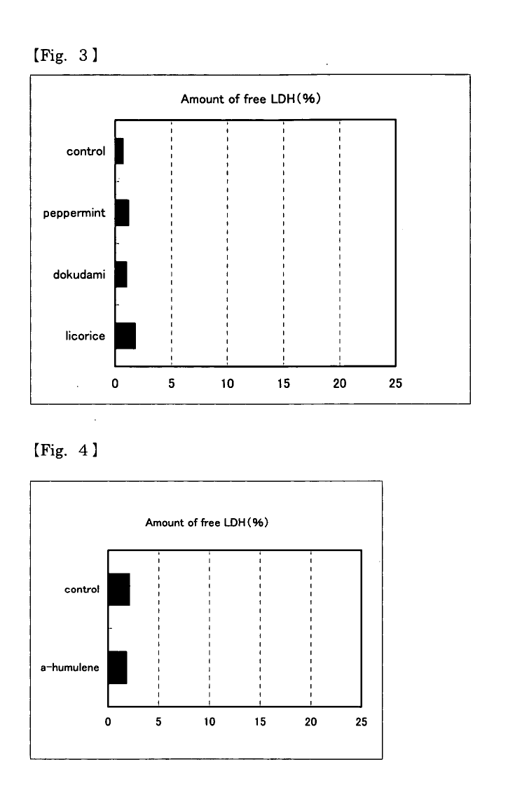

As seen from FIG. 3, each of the peppermint, dokudami (houttuynia herb), and licorice extracts did not differ from a control, and was therefore shown to produce no cytotoxicity. Furthermore, the effect of promoting IL-8 production exhibited by each of the peppermint, dokudami (houttuynia herb), and licorice extracts was shown to be independent of cytotoxicity.

### EXAMPLE 7

### Evaluation of Cytotoxicity by LDH (Lactate Dehydrogenase) Assay (2)

The cytotoxicity of α-humulene was evaluated with Caco-2 cells. The Caco-2 cells differentiated into small-intestinal epithelioid cells in the same way as in Example 4 were cultured for 24 hours in a medium containing 100 μg/ml α-humulene. Subsequently, the medium was removed, and the resulting cells were washed twice with PBS (−) (Nissui Co., Ltd.). The cells were further supplemented with 700 μl of PBS (−) and left undisturbed at 37° C. for 2 hours. Then, the PBS (−) was collected, and the cells were dissolved in 500 μl of 0.1 Triton X-100 (Nacalai Tesque, Inc.). LDH-Cytotoxic Test Wako (Wako Pure Chemical Industries, Ltd.) was used to measure LDH concentrations in the collected PBS (−) and cell lysate. The rate of free LDH (6) was calculated according to the equation below by using the LDH concentration in the collected PBS (—) as the amount of free LDH and the LDH concentration in the cell lysate as the amount of intracellular LDH.
Rate of free LDH(%)=(Amount of free LDH)/(Amount of free LDH+Amount of intracellular LDH)×100.

As seen from FIG. 4, the α-humulene did not differ from a control, and was therefore shown to produce no cytotoxicity. Furthermore, the effect of promoting IL-8 production exhibited by the α-humulene was shown to be independent of cytotoxicity.

### EXAMPLE 8

### Preparation of Capsule

A mixture was prepared in the proportion of 40 parts by weight of a peppermint extract obtained by the method described in Example 1, 30 parts by weight of sodium carboxymethylcellulose, 20 parts by weight of crystalline cellulose, and 10 parts by weight of vitamin C. The mixture was crushed and packed into a gelatin capsule to prepare a peppermint extract-containing capsule for food and drink use.

### EXAMPLE 9

### Preparation of Capsule

A dokudami (houttuynia herb) extract-containing capsule for food and drink use was prepared in the same way as in Example 8 except that the dokudami (houttuynia herb) extract described in Example 2 was used instead of a peppermint extract.

### EXAMPLE 10

### Preparation of Capsule

A licorice extract-containing capsule for food and drink use was prepared in the same way as in Example 8 except that the licorice extract described in Example 3 was used instead of a peppermint extract.

### INDUSTRIAL APPLICABILITY

According to the present invention, IL-8 production promoters derived from safe food materials as well as immunostimulants or preventive or treating agents of infectious disease comprising this IL-8 production promoter can be obtained. These IL-8 production promoters and compositions are useful in the stimulation of immune response and in the prevention or treatment of infectious disease, and can be utilized as foods and drinks such as foods with health claims (foods for specified health uses, foods with nutrient function claims), health foods, and dietary supplements, or as pharmaceutical drugs or quasi drugs.

1. An interleukin-8 production promoter comprising as an active ingredient at least one kind selected from the group consisting of a peppermint extract and a dokudami (houttuynia herb) extract.

2. An interleukin-8 production promoter comprising a licorice extract as an active ingredient.

3. An interleukin-8 production promoter comprising as an active ingredient at least one compound selected from the group consisting of (α-humulene represented by formula (1): 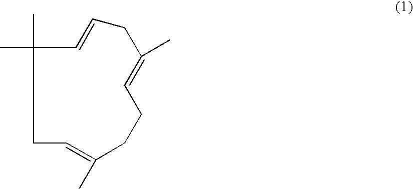
α-pinene represented by formula (2): 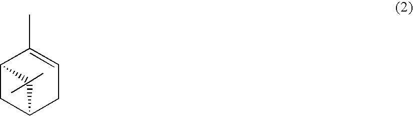
β-pinene represented by formula (3): 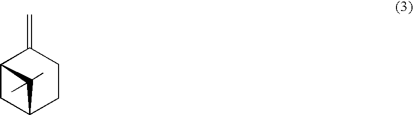
and L-menthol represented by formula (4): 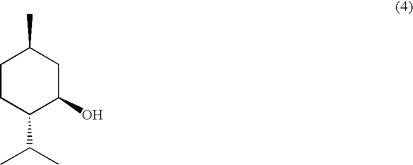
or a pharmaceutically acceptable salt thereof.

4. A composition for promoting interleukin production comprising an interleukin-8 production promoter according to any one of claims 1 to 3 as an active ingredient.

5. A composition for immunostimulation comprising an interleukin-8 production promoter according to any one of claims 1 to 3 as an active ingredient.

6. A composition for preventing or treating an infectious disease comprising an interleukin-8 production promoter according to any one of claims 1 to 3 as an active ingredient.

7. The composition according to any one of claim 4, wherein the composition is used for food and drink use.

8. A method for promoting interleukin production in mammal comprising administering an effective amount of at least one kind selected from the group consisting of a peppermint extract, a dokudami (houttuynia herb) extract, a licorice extract, a compound represented by formula (1), a compound represented by formula (2), a compound represented by formula (3), and a compound represented by formula (4) to mammal that need them.

9. A method for stimulating immunity in mammal comprising administering an effective amount of at least one kind selected from the group consisting of a peppermint extract, a dokudami (houttuynia herb) extract, a licorice extract, a compound represented by formula (1), a compound represented by formula (2), a compound represented by formula (3), and a compound represented by formula (4) to mammal that need them.

10. A method for preventing or treating an infectious disease in mammal comprising administering an effective amount of at least one kind selected from the group consisting of a peppermint extract, a dokudami (houttuynia herb) extract, a licorice extract, a compound represented by formula (1), a compound represented by formula (2), a compound represented by formula (3), and a compound represented by formula (4) to mammal that need them.

11. The composition according to claim 5, wherein the composition is used for food and drink use.

12. The composition according to claim 6, wherein the composition is used for food and drink use.
